(19) United States
(12) Patent Application Publication
(10) Pub. No.: US 2002/0028936 A1
(43) Pub. Date: Mar. 7, 2002

(54) 1,3-disubstituted indolin-2-ones for neoplasia

(76) Inventors: Gerhard Sperl, North Wales, PA (US); Xiaojing Wang, Livermore, CA (US); Gary A. Piazza, Doylestown, PA (US); Rifat Pamukcu, Spring House, PA (US)

Correspondence Address: Cell Pathways, Inc., 702 Electronic Drive, Horsham, PA, 19044, US

(21) Appl. No.: 09/966,003

(22) Filed: Sep. 28, 2001

Related U.S. Application Data
(63) Continuation of application No. 09/290,540, filed on Apr. 12, 1999, now abandoned.

(51) Int. Cl. C 07D 4 9/02

(52) U.S. Cl. 544/212; 544/333; 544/405; 546/281.1; 548/203; 548/252; 548/311.4; 548/364.4; 548/454; 548/527

(57) ABSTRACT

1,3-Disubstituted Indolin-2-Ones compounds are useful in the treatment of neoplasia.

This application is a Continuation of prior U.S. application Ser. No. 09/290,540 filed Apr. 12, 1999 entitled “1,3-Disubstituted Indolin-2-Ones for Neoplasia” which is incorporated herein by reference.

### TECHNICAL FIELD

This invention relates to compounds and methods for inducing or promoting apoptosis and for arresting uncontrolled neoplastic cell proliferation, methods that are specifically useful in the arresting and treatment of neoplasias, including precancerous and cancerous lesions.

### BACKGROUND OF THE INVENTION

Pharmaceuticals that are effective against early stage neoplasias comprise an emerging and expanding area of research and potential commercial development. Such pharmaceuticals can delay or arrest development of precancerous lesions into cancers. Each year in the United States alone, untold numbers of people develop precancerous lesions, which exhibit a strong statistically significant tendency to develop into malignant tumors, or cancer. Such lesions include lesions of the breast (that can develop into breast cancer), lesions of the skin (that can develop into malignant melanoma or basal cell carcinoma), colonic adenomatous polyps (that can develop into colon cancer), cervical displasia (cervical cancer) and other such neoplasms.

Such compounds and methods are particularly beneficial to sub-populations of patients who repeatedly develop precancerous lesions, and therefore have a statistically higher probability of getting cancer. Many cancer types (e.g., breast, colon, prostate etc.) have such patient sub-populations.

The search for drugs useful for treating and preventing neoplasias in their earliest stages is intensive because chemotherapy and surgery on cancer itself is often not effective, and current cancer chemotherapy has severe side effects. Such cancer-preventative compounds are also envisaged for recovered cancer patients who retain a risk of cancer reoccurrence, and even for cancer patients who would benefit from compounds that selectively induce apoptosis in neoplastic, but substantially not in normal cells.

Because it is believed that chronic administration of cancer-preventative pharmaceuticals is necessary to inhibit or arrest the development of neoplasia, standard cancer chemotherapeutic drugs are not considered appropriate drugs for cancer chemoprevention because whatever cancer preventative (as opposed to cancer-fighting) capabilities those drugs may possess do not outweigh their severe side effects. Most standard chemotherapeutics are now believed to kill cancer cells by inducing apoptosis (also sometimes referred to as “programmed cell death”). Apoptosis naturally occurs in many tissues in the body. Apoptosis plays a critical role in tissue homeostasis, that is, it ensures that the number of new cells produced are correspondingly offset by an equal number of cells that die. Apoptosis is especially pronounced in self-renewing tissues such as bone marrow, immune cells, gut, and skin. For example, the cells in the intestinal lining divide so rapidly that the body must eliminate cells after only three days to protect and prevent the overgrowth of the intestinal lining.

Standard chemotherapeutics promote apoptosis not only in cancer cells, but also in normal human tissues, and therefore have a particularly severe effect on tissues where apoptosis is especially pronounced (e.g. hair, gut and skin). The results of those effects include hair loss, weight loss, vomiting and bone marrow immune suppression. Thus, standard chemotherapeutics are inappropriate for cancer prevention, particularly if chronic administration is indicated.

Several non-steroidal anti-inflammatory drugs (“NSAIDs”), originally developed to treat arthritis, have shown effectiveness in inhibiting and eliminating colonic polyps. Polyps virtually disappear when the patients take the drug, particularly when the NSAID sulindac is administered. However, the continued prophylactic use of currently available NSAIDs, even in high colon cancer-risk patients, is still marked by severe side reactions that include gastrointestinal irritations, perforations, ulcerations and kidney toxicity believed to be produced by inhibition of prostaglandin synthetase activity (“PGE-2”). Such inhibition is a requirement for the NSAIDs anti-inflammatory action since elevated levels of PGE-2 are associated with inflammation. PGE-2 plays a protective function in the gastrointestinal tract, which is the reason such gastric side effects arise with chronic NSAID therapy, which is rarely indicated for arthritis sufferers, acute therapy being the norm for them. However, chronic administration of sulindac is important for high cancer-risk patients to eliminate and prevent future polyps which causes gastric side effects in many such patients. Once NSAID treatment is terminated due to such complications, the neoplasms return, particularly in high risk patients.

Compounds such as those disclosed in U.S. Pat. No. 5,643,959 have exhibited advantages in the treatment of neoplastic lesions since such compounds have been shown to induce apoptosis in neoplastic cells but not in normal cells in humans. Thus, the severe side effects due to induction of apoptosis in normal cells by conventional chemotherapeutics are avoided by these novel therapeutics (see, Van Stock, et al., Gastroenterology, 112 (4): A673, 1997). In addition, such compounds do not exhibit the gastric side effects associated with NSAIDs since such compounds do not substantially inhibit PGE-2. More potent compounds with such neoplasia specificity but without substantial PGE-2 activity are desirable.

### SUMMARY OF THE INVENTION

This invention represents potent compounds that induce apoptosis in neoplastic cells (but not substantially in normal cells), for treating patients with neoplastic lesions without substantially inhibiting PGE-2. This invention also involves methods for inducing such specific apoptosis in neoplastic cells by exposing such cells to a pharmacologically effective amount of those compounds described below to a patient in need of such treatment. Such compositions are effective in modulating apoptosis and modulating the growth of neoplasms, but are not suffering from the side effects of conventional chemotherapeutics and NSAIDs.

### DETAILED DESCRIPTION OF THE INVENTION

As discussed above, the present invention includes compounds of Formula I below

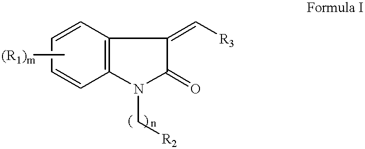

wherein

R₁ is selected from a group consisting of halogen, alkyl, alkanoyloxy, alkoxy, acylamino, amino, alkylamino, dialkylamino, dialkylaminoalkyl, sulfamyl, alkylthio, mercapto, hydroxy, hydroxyalkyl, alkenyl, alkenyloxy, alkylsulfinyl, alkylsulfonyl, dialkylsulfamyl, carboxyl, carbalkoxy, carbamido, haloalkyl, cycloalkyl, cyano or alkenyloxy;

R₂ is selected from a group consisting of substituted or unsubstituted aryl, wherein said aryl group is selected from the group consisting of phenyl, pyridyl, pyrimidyl, pyrazinyl, imidazolyl, indolyl, triazinyl, tetrazolyl, thiophenyl, furanyl, thiazolyl, pyrazolyl, or pyrrolyl, and wherein said substituents are one, two, three, four or five independently selected from the group consisting of halogen, alkyl, alkanoyloxy, alkoxy, acylamino, amino, alkylamino, dialkylamino, dialkylaminoalkyl, sulfamyl, alkylthio, mercapto, hydroxy, hydroxyalkyl, alkenyl, alkenyloxy, alkylsulfinyl, alkylsulfonyl, dialkylsulfamyl, carboxyl, carbalkoxy, carbamido, haloalkyl, cycloalkyl, cyano or alkenyloxy;

R₃ is a substituted or unsubstituted aryl, wherein said aryl group is selected from the group consisting of phenyl, pyridyl, pyrimidyl, pyrazinyl, imidazolyl, indolyl, triazinyl, tetrazolyl, thiophenyl, furanyl, thiazolyl, pyrazolyl, or pyrrolyl, and wherein said substituents are one, two, three, four, or five independently selected from the group consisting of halogen, alkyl, alkanoyloxy, alkoxy, acylamino, amino, alkylamino, dialkylamino, dialkylaminoalkyl, sulfamyl, alkylthio, mercapto, hydroxy, hydroxyalkyl, alkenyl, alkenyloxy, alkylsulfinyl, alkylsulfonyl, dialkylsulfamyl, carboxyl, carbalkoxy, carbamido, haloalkyl, cycloalkyl, cyano or alkenyloxy;

m is 0, 1, or 2;

n is 0, 1 or 2; and pharmaceutically acceptable salts thereof.

Preferred compounds of Formula I include those wherein

R₁ is selected from a group consisting of halogen, alkoxy, amino, dialkylamino, dialkylaminoalkyl, alkylthio, hydroxy, hydroxyalkyl, alkylsulfinyl, alkylsulfonyl, carboxyl, haloalkyl, or cyano;

R₂ is selected from a group consisting of substituted or unsubstituted aryl, wherein said aryl group is selected from the group consisting of phenyl, pyridyl, pyrimidyl, pyrazinyl, imidazolyl, triazinyl, pyrazolyl, or pyrrolyl, and wherein said substituents are one, two, or three independently selected from the group consisting of halogen, alkoxy, amino, dialkylamino, dialkylaminoalkyl, alkylthio, hydroxy, alkylsulfinyl, alkylsulfonyl, carboxyl, carbamido, haloalkyl, or cyano;

R₃ is a substituted or unsubstituted aryl, wherein said aryl group is selected from the group consisting of phenyl, pyridyl, pyrimidyl, pyrazinyl, imidazolyl, triazinyl, pyrazolyl, or pyrrolyl, and wherein said substituents are one, two or three independently selected from the group consisting of halogen, alkoxy, amino, dialkylamino, dialkylaminoalkyl, alkylthio, hydroxy, alkylsulfinyl, alkylsulfonyl, carboxyl, carbamido, haloalkyl, or cyano;

m is 0, or 1;

n is 1; and pharmaceutically acceptable salts thereof.

The most preferred group of compounds of this invention include those more preferred compounds of Formula I where

R₁ is selected from a group consisting of halogen, alkoxy, dialkylamino, dialkylaminoalkyl, alkylthio, alkylsulfinyl, alkylsulfonyl, or cyano;

R₂ is substituted aryl, wherein said aryl group is selected from the group consisting of phenyl, pyridyl, pyrimidyl, pyrazinyl, or triazinyl, and wherein said substituents are one or three independently selected from the group consisting of halogen, alkoxy, dialkylamino, dialkylaminoalkyl, alkylthio, alkylsulfonyl, or carboxyl;

R₃ is a substituted aryl, wherein said aryl group is selected from the group consisting of phenyl, pyridyl, pyrimidyl, pyrazinyl, or triazinyl, and wherein said substituents are one or three independently selected from the group consisting of halogen, alkoxy, dialkylamino, dialkylaminoalkyl, alkylthio, alkylsulfonyl, or carboxyl;

m is 1;

n is 1; and pharmaceutically acceptable salts thereof.

The present invention is also a method of treating individuals with neoplastic lesions by administering a pharmacologically effective amount of an enterically coated pharmaceutical composition that includes compounds of this invention.

Preferably, such compounds are administered without therapeutic amounts of an NSAID.

Also, the present invention is a method of inhibiting the growth of neoplastic cells by exposing the cells to an effective amount of compounds of Formula I, wherein R₁, R₂, R₃, m and n are defined as above.

In still another form, the invention is a method of inducing apoptosis in human cells by exposing those cells to an effective amount of compounds of Formula I, wherein R₁ through R₃ and n are defined as above where such cells are sensitive to these compounds.

Additionally, in yet another form, the invention is a method of treating a patient having a disease which would benefit from regulation of apoptosis by treating the patient with an effective amount of compounds of Formula I, wherein R₁, R₂, m, n are defined as above. The regulation of apoptosis is believed to play an important role in diseases associated with abnormalities of cellular growth patterns such as benign prostatic hyperplasia, neurodegenerative diseases such as Parkinson's disease, autoimmune diseases including multiple sclerosis and rheumatoid arthritis, infectious diseases such as AIDS, and other diseases, as well.

As used herein, the term “precancerous lesion” includes syndromes represented by abnormal neoplastic, including dysplastic, changes of tissue. Examples include dysplasic growths in colonic, breast, bladder or lung tissues, or conditions such as dysplastic nevus syndrome, a precursor to malignant melanoma of the skin. Examples also include, in addition to dysplastic nevus syndromes, polyposis syndromes, colonic polyps, precancerous lesions of the cervix (i.e., cervical dysplasia), esophagus, prostatic dysplasia, bronchial dysplasia, breast, bladder and/or skin and related conditions (e.g., actinic keratosis), whether the lesions are clinically identifiable or not.

As used herein, the term “cancerous” refers to lesions that are malignant. Examples include malignant melanomas, breast cancer, prostate cancer and colon cancer.

As used herein, the term “neoplasm” refers to both precancerous and cancerous lesions and hyperplasia.

As used herein, the term “halo” or “halogen” refers to chloro, bromo, fluoro and iodo groups, and the term “alkyl” refers to straight, branched or cyclic alkyl groups and to substituted aryl alkyl groups. The term “lower alkyl” refers to C₁ to C₈ alkyl groups.

The term “lower alkoxy” refers to alkoxy groups having from 1 to 8 carbons, including straight, branched or cyclic arrangements.

The term “pharmaceutically acceptable salt” refers to non-toxic acid addition salts and alkaline earth metal salts of the compounds of Formula I. The salts can be prepared in situ during the final isolation and purification of such compounds, or separately by reacting the free base or acid functions with a suitable organic acid or base, for example. Representative acid addition salts include the hydrochloride, hydrobromide, sulfate, bisulfate, acetate, valerate, oleate, palmatate, stearate, laurate, borate, benzoate, lactate, phosphate, tosylate, mesylate, citrate, maleate, fumarate, succinate, tartrate, glucoheptonate, lactobionate, lauryl sulfate salts and the like. Representative alkali and alkaline earth metal salts include the sodium, calcium, potassium and magnesium salts.

It will be appreciated that certain compounds of Formula I possess an asymmetric carbon atom and are thus capable of existing as enantiomers. Unless otherwise specified, this invention includes such enantiomers, including any racemates. The separate enaniomers may be synthesized from chiral starting materials, or the racemates can be resolved by conventional procedures that are well known in the art of chemistry such as chiral chromatography, fractional crystallization of diastereomeric salts and the like.

Compounds of Formula I also can exist as geometrical isomers (Z and E).

Compounds of this invention may be formulated into pharmaceutical compositions together with pharmaceutically acceptable carriers for oral administration in solid or liquid form, or for rectal or topical administration, although carriers for oral administration are most preferred.

Pharmaceutically acceptable carriers for oral administration include capsules, tablets, pills, powders, troches and granules. In such solid dosage forms, the carrier can comprise at least one inert diluent such as sucrose, lactose or starch. Such carriers can also comprise, as is normal practice, additional substances other than diluents, e.g., lubricating agents such as magnesium stearate. In the case of capsules, tablets, troches and pills, the carriers may also comprise buffering agents. Carriers such as tablets, pills and granules can be prepared with enteric coatings on the surfaces of the tablets, pills or granules. Alternatively, the enterically coated compound can be pressed into a tablet, pill, or granule, and the tablet, pill or granules for administration to the patient. Preferred enteric coatings include those that dissolve or disintegrate at colonic pH such as shellac or Eudraget S.

Pharmaceutically acceptable carriers include liquid dosage forms for oral administration, e.g., pharmaceutically acceptable emulsions, solutions, suspensions, syrups and elixirs containing inert diluents commonly used in the art, such as water. Besides such inert diluents, compositions can also include adjuvants such as wetting agents, emulsifying and suspending agents, and sweetening, flavoring and perfuming agents.

Pharmaceutically acceptable carriers for topical administration include DMSO, alcohol or propylene glycol and the like that can be employed with patches or other liquid-retaining material to hold the medicament in place on the skin so that the medicament will not dry out.

Pharmaceutically acceptable carriers for rectal administration are preferably suppositories that may contain, in addition to the compounds of this invention excipients such as cocoa butter or a suppository wax, or gel.

The pharmaceutically acceptable carrier and compounds of this invention are formulated into unit dosage forms for administration to a patient. The dosage levels of active ingredient (i.e., compounds of this invention) in the unit dosage may be varied so as to obtain an amount of active ingredient effective to achieve lesion-eliminating activity in accordance with the desired method of administration (i.e., oral or rectal). The selected dosage level therefore depends upon the nature of the active compound administered, the route of administration, the desired duration of treatment, and other factors. If desired, the unit dosage may be such that the daily requirement for active compound is in one dose, or divided among multiple doses for administration, e.g., two to four times per day.

The pharmaceutical compositions of this invention are preferably packaged in a container (e.g., a box or bottle, or both) with suitable printed material (e.g., a package insert) containing indications, directions for use, etc.

There is one general scheme for producing compounds useful in this invention.

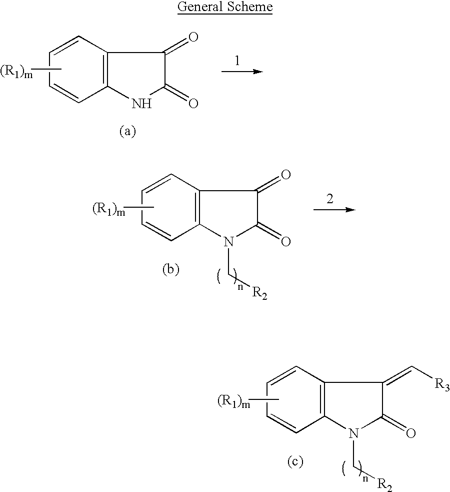

The isatin (a) is subjected to sodium hydride and a halide (R₂—(CH₂)ₙ—Hal) to give the 1-substituted isatin (b) (reaction 1). A Wittig reaction with a base such as lithium ethanolate or n-butyl lithium and a substituted triphenylphosphonium bromide (R₃—CH₂—PPh₃⁺Br⁻) yields the 1-substituted indolin-2-one (c) (reaction 2).

The foregoing may be better understood from the following examples that are presented for the purposes of illustration and are not intended to limit the scope of the invention. As used in the following examples, the references to substituents such as R₁ R₂, etc. refer to the corresponding substituents in Formula I above.

### EXAMPLE 1

### (Z, E) 1-(4-Fluorobenzyl)-3-Benzylidene-Indolin-2-One

A.) (Z, E) 1-(4-Fluorobenzyl)indolin-2,3-dione

Isatin (3.4 mmol, 0.5 g) in dry dimethylformamide (5 ml) at 0° C. is charged with potassium hydride (6.8 mmol, 0.27 g). 4-Fluorobenzyl bromide (5.1 mmol) is added at 0° C., and the mixture is stirred overnight. The mixture is poured into ice water/10% HCl, is filtrated and is washed with water. The solid is dissolved with ethyl acetate, is extracted with water and is dried with Na₂SO₄ to yield the title compound. B.) (Z, E) 1-(4-Fluorobenzyl)-3-benzylidene-indolin-2-one

Lithium ethanolate (1 molar solution in ethanol, 1.2 ml) is added to a suspension of benzyl triphenylphosphonium bromide (1.2 mmole) in ethanol (7 ml). After 10 minutes, 1-(4-Fluorobenzyl)indolin-2,3-dione (1 mmole) is added. The mixture is stirred at room temperature over night. The reaction mixture is acidified with 10% HCl, and extracted with ethyl acetate. The organic phase is dried over Na₂SO₄. The solvent is evaporated to yield a mixture of the two geometrical isomers. The two isomers are separated with flash chromatography (eluent hexane:THF=9:1). (R₃=phenyl, R₂=4-fluorophenyl, m=0, n=1). The isomer pure compounds convert back to an isomer mixture in solutions.

### EXAMPLE 2

Similarly, when benzyl bromide, 3-chlorobenzyl bromide, 3-fluorobenzyl bromide, 2-chloro-6-fluorobenzyl chloride, 4-chlorobenzyl chloride, α-bromo-3,4-difluoro toluene, 2-chlorobenzyl bromide, 2-chlorophenyl bromide, α-bromo-2,6-difluoro toluene, 2,3,4,5,6-pentafluorobenzyl bromide, α-bromo-2,4-difluoro toluene, 2-bromophenol, 3-bromophenol, 4-bromophenol, 2-bromopyridine, 3-bromopyridine, 4-bromopyridine, 5-bromopyrimidine, 2-chloro-pyrimidine chloropyrazine, 5-bromoindol, 7-bromoindol, 2-bromothiophene, 3-bromothiophene, 3-bromofuran, 2-bromothiazole, 4-bromopyrazole instead of 4-fluorobenzyl bromide, the corresponding Z, E mixtures of N-substituted 3-benzylidene-indolin-2-ones are obtained: Z, E mixture of: 1-benzyl-3-benzylidene-indolin-2-one, 1-(3-chlorobenzyl)-3-benzylidene-indolin-2-one, 1-(3-fluorobenzyl)-3-benzylidene-indolin-2-one, 1-(2-chloro-6-fluoro)-3-benzylidene-indolin-2-one, 1-(4-chloro)-3-benzylidene-indolin-2-one, 1-(3,4-difluoro-tolyl)-3-benzylidene-indolin-2-one, 1-(2-chlorobenzyl)-3-benzylidene-indolin-2-one, 1-(2-chlorophenyl)-3-benzylidene-indolin-2-one, 1-(2,6-difluorotolyl)-3-benzylidene-indolin-2-one, 1-(2,3,4,5,6-pentafluorobenzyl)-3-benzylidene-indolin-2-one, 1-(2,4-difluorotoyl)-3-benzylidene-indolin-2-one, 1-(2-hydroxyphenyl)-3-benzylidene-indolin -2-one, 1-(3-hydroxyphenyl)-3-benzylidene-indolin-2-one, 1-4-hydroxyphenyl) -3-benzylidene-indolin-2-one, 1-(2-pyridyl)-3-benzylidene-indolin-2-one, 1-(3-pyridyl)-3-benzylidene-indolin-2-one, 1-(4-pyridyl)-3-benzylidene-indolin-2-one, 1-(5-pyrimidyl)-3-benzylidene-indolin-2-one, 1-(2-pyrimidyl)-3-benzylidene-indolin-2-one, 1-pyrazinyl-3-benzylidene-indolin-2-one, 1-(5-indolyl)-3-benzylidene-indolin-2-one, 1-(7-indolyl)-3-benzylidene-indolin-2-one, 1-(2-thiophenyl)-3-benzylidene-indolin-2-one, 1-(3-thiophenyl)-3-benzylidene-indolin-2-one, 1-(3-furanyl)-3-benzylidene-indolin-2-one, 1-(2-thiazolyl)-3-benzylidene-indolin-2-one, and 1-(4-pyrazolyl)-3-benzylidene-indolin-2-one.

### EXAMPLE 3

Example 2, above, is repeated with 5-bromoisatin instead of isatin to obtain the corresponding 5-bromo-N-substituted 3-benzylidene-indolin-2-ones.

### EXAMPLE 4

Example 2, above, is repeated with 5-methylisatin instead of isatin to obtain the corresponding 5-methyl-N-substituted 3-benzylidene-indolin-2-ones.

### EXAMPLE 5

Example 2, above, is repeated with 5-chloroisatin instead of isatin to obtain the corresponding 5-chloro-N-substituted 3-benzylidene-indolin-2-ones.

### EXAMPLE 6

Example 2, above, is repeated with 5-iodoisatin instead of isatin to obtain the corresponding 5-iodo-N-substituted 3-benzylidene-indolin-2-ones.

### EXAMPLE 7

Example 2, above, is repeated with 5-fluoroisatin instead of isatin to obtain the corresponding 5-fluoro-N-substituted 3-benzylidene-indolin-2-ones.

### EXAMPLE 8

Example 2, above, is repeated with 7-chloroisatin instead of isatin to obtain the corresponding 7-chloro-N-substituted 3-benzylidene-indolin-2-ones.

### EXAMPLE 9

Example 2, above, is repeated with 7-methylisatin instead of isatin to obtain the corresponding 7-methyl-N-substituted 3-benzylidene-indolin-2-ones.

### EXAMPLE 10

Example 2, above, is repeated with 5,7-dibromoisatin instead of isatin to obtain the corresponding 5,7-dibromo-N-substituted 3-benzylidene-indolin-2-ones.

### EXAMPLE 11

Example 2, above, is repeated with 5,7-dichloroisatin instead of isatin to obtain the corresponding 5,7-dichloro-N-substituted 3-benzylidene-indolin-2-ones.

### EXAMPLE 12

Example 2, above, is repeated with 4,7-dichloroisatin instead of isatin to obtain the corresponding 4,7-dichloro-N-substituted 3-benzylidene-indolin-2-ones.

### EXAMPLE 13

Example 2, above, is repeated with 4-chloro-7-methoxy isatin instead of isatin to obtain the corresponding 4-chloro-7-methoxy-N-substituted 3-benzylidene-indolin-2-ones.

### EXAMPLE 14

Example 2, above, is repeated with 4-chloro-7-methyl isatin instead of isatin to obtain the corresponding 4-chloro-7-methyl-N-substituted 3-benzylidene-indolin-2-ones.

EXAMPLE 15

Example 2, above, is repeated with 4,7-dimethyl isatin instead of isatin to obtain the corresponding 4,7-dimethyl-N-substituted 3-benzylidene-indolin-2-ones.

### EXAMPLE 16

Example 2, above, is repeated with 5-chloro-7-methyl isatin instead of isatin to obtain the corresponding 5-chloro-7-methyl-N-substituted 3-benzylidene-indolin-2-ones.

### EXAMPLE 17

Example 2, above, is repeated with 5,7-dimethyl isatin instead of isatin to obtain the corresponding 5,7-dimethyl-N-substituted 3-benzylidene-indolin-2-ones.

### EXAMPLE 18

Example 2, above, is repeated with 6-chloro-7-methyl isatin instead of isatin to obtain the corresponding 6-chloro-7-methyl-N-substituted 3-benzylidene-indolin-2-ones.

### EXAMPLE 19

Example 2, above, is repeated with 6,7-dimethyl isatin instead of isatin to obtain the corresponding 6,7-dimethyl-N-substituted 3-benzylidene-indolin-2-ones.

### EXAMPLE 20

Example 2, above, is repeated with 6-chloro isatin instead of isatin to obtain the corresponding 6-chloro-N-substituted 3-benzylidene-indolin-2-ones.

### EXAMPLE 21

Example 2, above, is repeated with 4,5,7-trichloro isatin instead of isatin to obtain the corresponding 4,5,7-trichloro-N-substituted 3-benzylidene-indolin-2-ones.

### EXAMPLE 22

Example 2, above, is repeated with 4-chloro isatin instead of isatin to obtain the corresponding 4-chloro-N-substituted 3-benzylidene-indolin-2-ones.

### EXAMPLE 23

Example 2, above, is repeated with 5-methoxy isatin instead of isatin to obtain the corresponding 5-methoxy-N-substituted 3-benzylidene-indolin-2-ones.

### EXAMPLE 24

Example 2, above, is repeated with 4,5-dimethyl isatin instead of isatin to obtain the corresponding 4,5-dimethyl-N-substituted 3-benzylidene-indolin-2-ones.

### EXAMPLE 25

Example 2, above, is repeated with 4,5-dichloro isatin instead of isatin to obtain the corresponding 4,5-dichloro-N-substituted 3-benzylidene-indolin-2-ones.

### EXAMPLE 26

Example 2, above, is repeated with 5-trifluoromethoxy isatin instead of isatin to obtain the corresponding 5-trifluoromethoxy-N-substituted 3-benzylidene-indolin-2-ones.

### EXAMPLE 27

Example 2, above, is repeated with 7-methoxy isatin instead of isatin to obtain the corresponding 7-methoxy-N-substituted 3-benzylidene-indolin-2-ones.

### EXAMPLE 28

Example 2, above, is repeated with 6-methoxy isatin instead of isatin to obtain the corresponding 6-methoxy-N-substituted 3-benzylidene-indolin-2-ones.

### BIOLOGICAL EFFECTS

(A) Growth Inhibition

The compound of Example No. 1 was assayed for their growth inhibitory activity on the human colon carcinoma cell line, HT-29 obtained from ATCC (Rockville, Md.), to ascertain the degree of growth inhibition. Growth inhibition of this cell line is indicative of a benefit on precancerous lesions and neoplasms. The cell line and growth inhibition assay employed for such experiments are well characterized, and are used to evaluate the anti-neoplastic properties of substances. The assay is used by the United States National Cancer Institute in its screening program for new anti-cancer drugs.

Drug stock solutions were made in 100% DMSO and were then diluted with RPMI media for cell culture testing. All drug solutions were prepared fresh on the day of testing. The cultured cells were obtained at passage #99 and grown in RPMI media supplemented with 5% fetal calf serum, and 2 mM glutamine, 100 U/ml penicillin, 100 U/ml streptomycin, and 0.25 μg/ml amphotericin. The cultures were maintained in a humidified atmosphere of 95% air and 5% CO₂ at 37° C. The cultures were passaged at preconfluent densities using a solution of 0.05% trypsin and 0.53 mM EDTA. Cells were plated at 1000 cells/well for 96 well flat-bottom microtiter plates.

Tumor cell growth inhibition was assessed using the Sulforhodamine B (SRB) protein binding assay. In this assay, tumor cells were plated in 96-well plates and treated with drug-containing media for six days (continuous exposure). For each plate, 6 wells were designated as no treatment controls, six wells as vehicle (0.1% DMSO) controls, and the remaining wells for drug dilutions with three wells per drug concentration. At the end of the exposure period, the cells were fixed and stained with sulforhodamine B, a protein binding dye. The dye was then solubilized, and the optical density of the resulting solution was determined on a 96-well plate reader. The mean dye intensity of the treated wells was then divided by the mean dye intensity in the control wells (6 wells of each) to determine the effect of the drug on the cells. Dye intensity is proportional to the number of cells or amount of protein per well. IC₅₀ value was obtained graphically by connecting the mean values for each drug concentration tested. This value is equivalent to the concentration of drug needed to inhibit tumor cell growth by 50%. Each experiment included at least three wells per drug concentration. Concentration was plotted on a log scale on the X-axis. IC₅₀ value obtained for the compound of Example 1 is approximately 7.3 μM.

(B) Cyclooxygenase (COX) Inhibition

COX catalyzes the formation of prostaglandins and thromboxane by the oxidative metabolism of arachidonic acid. The compound of Example 1 of this invention, as well as a positive control, (sulindac sulfide) was evaluated to determine whether it inhibited purified cyclooxygenase Type I (see Table 1 below).

The compounds of this invention were evaluated for inhibitory effects on purified COX. The COX was purified from ram seminal vesicles, as described by Boopathy, R. and Balasubramanian, J., 239:371-377, 1988. COX activity was assayed as described by Evans, A. T., et al., “Actions of Cannabis Constituents on Enzymes Of Arachidonate Metabolism Anti-Inflammatory Potential,” Biochem. Pharmacol., 36:2035-2037, 1987. Briefly, purified COX was incubated with arachidonic acid (100 μM) for 2.0 min at 37° C. in the presence or absence of test compounds. The assay was terminated by the addition of TCA, and COX activity was determined by absorbance at 530 nm.

TABLE 1

| | COX I |
|---|---|
| EXAMPLE | % Inhibition (100 μM) |
| Sulindac sulfide | 86 |
| Example 1 | <25 |

(C) Apoptosis

Apoptosis was measured based on the amount of fragmented DNA contained in cell lysates. Briefly, SW-480 colon adenocarcinoma cells were plated in 96-well microtitre plates (“MTP”) at a density of 10K cells/well in 180 μl and were incubated for 24 hrs. Cells were then treated with 20 μl aliquots of appropriately diluted compound, and allowed to incubate for an additional 48 hrs.

After the incubation, samples were prepared according to the following steps. The MTP was centrifuged (15 min., 1000 rpm) and the supernatant was carefully removed by fast inversion of the MTP. The cell pellets in each well were resuspended in 200 μl lysis buffer and incubated for 45 min. at room temperature to lyse the cells. The lysates were then centrifuged (15 min., 1000 rpm) and 20 μl aliquots of the supernatant (=cytoplasmic fraction) were transferred into the streptavidin coated MTP for analysis. Care was taken not to shake the lysed pellets in the MTP (=cell nucleii containing high molecular weight, unfragmented DNA). Samples were analyzed immediately, because storage at 4° C. or −20° C. reduces the ELISA-signals.

Samples were then processed according to a DNA fragmentation assay protocol, and dose-response curves were generated based on optical density readings. Quantification of DNA was done by a commercially available photometric enzyme-immunoassay manufactured by Mannheim-Boehringer under the name “Cell Death Detection ELISA^{plus}”. The assay is based on a quantitative sandwich-enzyme-immunoassay-principle using mouse monoclonal antibodies directed against DNA and histones, respectively. This allows the specific determination of mono and oligonucleosomes in the cytoplasmatic fraction of cell lysates. In brief, the assay procedure is as follows. The sample (cell-lysate, serum, culture-supernatant etc.) is placed into a streptavidin-coated MTP. Subsequently, a mixture of anti-histone-biotin and anti-DNA-POD is followed by incubation for 2 hours. During the incubation period, the anti-histone antibody binds to the histone-component of the nucleosomes and simultaneously fixes the immunocomplex to the streptavidin-coated MTP via its biotinylation. Additionally, the anti-DNA-POD antibody reacts with the DNA component of the nucleosomes. After removal of unbound antibodies by a washing step, the amount of nucleosomes is quantified by the POD retained in the immunocomplex. POD is determined photometrically with ABTS® (2,2′-Azino-di[3-ethylbenzthiazolin-sulfonate) as substrate. For example 1 a fold stimulation of 6 at 100 μM was found.

The compounds of this invention can be formulated with pharmaceutically acceptable carriers into unit dosage forms in a conventional manner so that the patient in need of therapy for precancerous lesions can periodically (e.g., once or more per day) take a compound according to the methods of this invention. The exact initial dose of the compounds of this invention can be determined with reasonable experimentation. One skilled in the art should understand that the initial dosage should be sufficient to achieve a blood plasma concentration approaching a percentage of the IC₅₀ value of the compound, with the percentage depending on the chemopreventative or chemotherapeutic indication. The initial dosage calculation would also take into consideration several factors, such as the formulation and mode of administration, e.g. oral or intravenous, of the particular compound. For example, assuming a patient with an average circulatory system volume of about four liters, based on the IC₅₀ values for compounds of this invention, one would calculate a dosage of from about 0.5-500 mg of such compounds for intravenous administration to achieve a systemic circulatory concentration equivalent to the IC₅₀ concentration.

It will be understood that various changes and modifications can be made in the details of procedure, formulation and use without departing from the spirit of the invention, especially as defined in the following claims.

We claim:

1. A compound of formula I 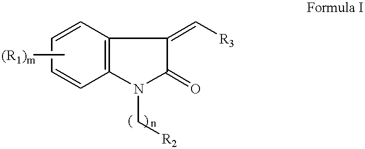
wherein
R₁ is selected from a group consisting of halogen, alkyl, alkanoyloxy, alkoxy, acylamino, amino, alkylamino, dialkylamino, dialkylaminoalkyl, sulfamyl, alkylthio, mercapto, hydroxy, hydroxyalkyl, alkenyl, alkenyloxy, alkylsulfinyl, alkylsulfonyl, dialkylsulfamyl, carboxyl, carbalkoxy, carbamido, haloalkyl, cycloalkyl, cyano or alkenyloxy;
R₂ is selected from a group consisting of substituted or unsubstituted aryl, wherein said aryl group is selected from the group consisting of phenyl, pyridyl, pyrimidyl, pyrazinyl, imidazolyl, indolyl, triazinyl, tetrazolyl, thiophenyl, furanyl, thiazolyl, pyrazolyl, or pyrrolyl, and wherein said substituents are one to five independently selected from the group consisting of halogen, alkyl, alkanoyloxy, alkoxy, acylamino, amino, alkylamino, dialkylamino, dialkylaminoalkyl, sulfamyl, alkylthio, mercapto, hydroxy, hydroxyalkyl, alkenyl, alkenyloxy, alkylsulfinyl, alkylsulfonyl, dialkylsulfamyl, carboxyl, carbalkoxy, carbamido, haloalkyl, cycloalkyl, cyano or alkenyloxy;
R₃ is a substituted or unsubstituted aryl, wherein said aryl group is selected from the group consisting of phenyl, pyridyl, pyrimidyl, pyrazinyl, imidazolyl, indolyl, triazinyl, tetrazolyl, thiophenyl, furanyl, thiazolyl, pyrazolyl, or pyrrolyl, and wherein said substituents are one to or five independently selected from the group consisting of halogen, alkyl, alkanoyloxy, alkoxy, acylamino, amino, alkylamino, dialkylamino, dialkylaminoalkyl, sulfamyl, alkylthio, mercapto, hydroxy, hydroxyalkyl, alkenyl, alkenyloxy, alkylsulfinyl, alkylsulfonyl, dialkylsulfamyl, carboxyl, carbalkoxy, carbamido, haloalkyl, cycloalkyl, cyano or alkenyloxy;
m is 0, 1, or 2;
n is 0, 1 or 2;
and pharmaceutically acceptable salts thereof.

2. The compound of claim 1 wherein R₁ is selected from a group consisting of halogen, alkoxy, amino, dialkylamino, dialkylaminoalkyl, alkylthio, hydroxy, hydroxyalkyl, alkylsulfinyl, alkylsulfonyl, carboxyl, haloalkyl, or cyano.

3. The compound of claim 2 wherein R₁ is selected from a group consisting of halogen, alkoxy, dialkylamino, dialkylaminoalkyl, alkylthio, alkylsulfinyl, alkylsulfonyl, or cyano.

4. The compound of claim 1 wherein R₂ is selected from the group consisting of substituted or unsubstituted aryl, wherein said aryl group is selected from the group consisting of phenyl, pyridyl, pyrimidyl, pyrazinyl, imidazolyl, triazinyl, pyrazolyl, or pyrrolyl, and wherein said substituents are one to three independently selected from the group consisting of halogen, alkoxy, amino, dialkylamino, dialkylaminoalkyl, alkylthio, hydroxy, alkylsulfinyl, alkylsulfonyl, carboxyl, carbamido, haloalkyl, or cyano.

5. The compound of claim 3 wherein R₂ is substituted aryl, wherein said aryl group is selected from the group consisting of phenyl, pyridyl, pyrimidyl, pyrazinyl, or triazinyl, and wherein said substituents are one or three independently selected from the group consisting of halogen, alkoxy, dialkylamino, dialkylaminoalkyl, alkylthio, alkylsulfonyl, or carboxyl.

6. The compound of claim 1 wherein R₃ is selected from a group consisting of substituted or unsubstituted aryl, wherein said aryl group is selected from the group consisting of phenyl, pyridyl, pyrimidyl, pyrazinyl, imidazolyl, triazinyl, pyrazolyl, or pyrrolyl, and wherein said substituents are one to three independently selected from the group consisting of halogen, alkoxy, amino, dialkylamino, dialkylaminoalkyl, alkylthio, hydroxy, alkylsulfinyl, alkylsulfonyl, carboxyl, carbamido, haloalkyl, or cyano.

7. The compound of claim 5 wherein R₃ is substituted aryl, wherein said aryl group is selected from the group consisting of phenyl, pyridyl, pyrimidyl, pyrazinyl, or triazinyl, and wherein said substituents are one or three independently selected from the group consisting of halogen, alkoxy, dialkylamino, dialkylaminoalkyl, alkylthio, alkylsulfonyl, or carboxyl.

8. The compound of claim 1 where m is 0 or 1.

9. The compound of claim 7 where m is 1.

10. The compound of claim 9 where n is 1.

11. A pharmaceutical composition comprising: a pharmaceutically acceptable carrier and a compound of the formula 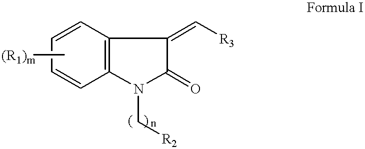
wherein
R₁ is selected from a group consisting of halogen, alkyl, alkanoyloxy, alkoxy, acylamino, amino, alkylamino, dialkylamino, dialkylaminoalkyl, sulfamyl, alkylthio, mercapto, hydroxy, hydroxyalkyl, alkenyl, alkenyloxy, alkylsulfinyl, alkylsulfonyl, dialkylsulfamyl, carboxyl, carbalkoxy, carbamido, haloalkyl, cycloalkyl, cyano or alkenyloxy;
R₂ is selected from a group consisting of substituted or unsubstituted aryl, wherein said aryl group is selected from the group consisting of phenyl, pyridyl, pyrimidyl, pyrazinyl, imidazolyl, indolyl, triazinyl, tetrazolyl, thiophenyl, furanyl, thiazolyl, pyrazolyl, or pyrrolyl, and wherein said substituents are one to five independently selected from the group consisting of halogen, alkyl, alkanoyloxy, alkoxy, acylamino, amino, alkylamino, dialkylamino, dialkylaminoalkyl, sulfamyl, alkylthio, mercapto, hydroxy, hydroxyalkyl, alkenyl, alkenyloxy, alkylsulfinyl, alkylsulfonyl, dialkylsulfamyl, carboxyl, carbalkoxy, carbamido, haloalkyl, cycloalkyl, cyano or alkenyloxy;
R₃ is selected from a group consisting of substituted or unsubstituted aryl, wherein said aryl group is selected from the group consisting of phenyl, pyridyl, pyrimidyl, pyrazinyl, imidazolyl, indolyl, triazinyl, tetrazolyl, thiophenyl, furanyl, tbiazolyl, pyrazolyl, or pyrrolyl, and wherein said substituents are one to five independently selected from the group consisting of halogen, alkyl, alkanoyloxy, alkoxy, acylamino, amino, alkylamino, dialkylamino, dialkylaminoalkyl, sulfamyl, alkylthio, mercapto, hydroxy, hydroxyalkyl, alkenyl, alkenyloxy, alkylsulfinyl, alkylsulfonyl, dialkylsulfamyl, carboxyl, carbalkoxy, carbamido, haloalkyl, cycloalkyl, cyano or alkenyloxy;
m is 0, 1, or 2;
n is 0, 1 or 2; and
pharmaceutically acceptable salts thereof.

12. The pharmaceutical composition of claim 11 wherein R₁ is selected from a group consisting of halogen, alkoxy, amino, dialkylamino, dialkylaminoalkyl, alkylthio, hydroxy, hydroxyalkyl, alkylsulfinyl, alkylsulfonyl, carboxyl, haloalkyl, or cyano.

13. The pharmaceutical composition of claim 12 wherein R₁ is selected from a group consisting of halogen, alkoxy, dialkylamino, dialkylaminoalkyl, alkylthio, alkylsulfinyl, alkylsulfonyl, or cyano.

14. The pharmaceutical composition of claim 11 wherein R₂ is selected from a group consisting of substituted or unsubstituted aryl, wherein said aryl group is selected from the group consisting of phenyl, pyridyl, pyrimidyl, pyrazinyl, imidazolyl, triazinyl, pyrazolyl, or pyrrolyl, and wherein said substituents are one to three independently selected from the group consisting of halogen, alkoxy, amino, alkylamino, dialkylamino, dialkylaminoalkyl, alkylthio, hydroxy, alkylsulfinyl, alkylsulfonyl, carboxyl, carbamido, haloalkyl, or cyano.

15. The pharmaceutical composition of claim 13 wherein R₂ is substituted aryl, wherein said aryl group is selected from the group consisting of phenyl, pyridyl, pyrimidyl, pyrazinyl, or triazinyl, and wherein said substituents are one or three independently selected from the group consisting of halogen, alkoxy, dialkylamino, dialkylaminoalkyl, alkylthio, alkylsulfonyl, or carboxyl.

16. The pharmaceutical composition of claim 11 where R₃ is a substituted or unsubstituted aryl, wherein said aryl group is selected from the group consisting of phenyl, pyridyl, pyrimidyl, pyrazinyl, imidazolyl, triazinyl, pyrazolyl, or pyrrolyl, and wherein said substituents are one to three independently selected from the group consisting of halogen, alkoxy, amino, dialkylamino, dialkylaminoalkyl, alkylthio, hydroxy, alkylsulfinyl, alkylsulfonyl, carboxyl, carbamido, haloalkyl, or cyano.

17. The pharmaceutical composition of claim 15 where R₃ is a substituted aryl, wherein said aryl group is selected from the group consisting of phenyl, pyridyl, pyrimidyl, pyrazinyl, triazinyl, and wherein said substituents are one or three independently selected from the group consisting of halogen, alkoxy, or, dialkylamino, dialkylaminoalkyl, alkylthio, alkylsulfonyl, or carboxyl.

18. The pharmaceutical composition of claim 11 where m is 0 or 1.

19. The pharmaceutical composition of claim 17 where m is 1.

20. The pharmaceutical composition of claim 19 where n is 1.

21. A method of treating a patient having neoplasia comprising:
administering a pharmacologically effective amount of a compound of formula I to a patient with a neoplasia sensitive to such a compound 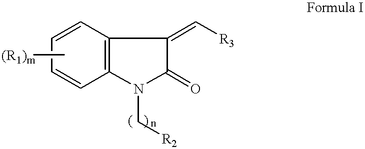
wherein
R₁ is selected from a group consisting of halogen, alkyl, alkanoyloxy, alkoxy, acylamino, amino, alkylamino, dialkylamino, dialkylaminoalkyl, sulfamyl, alkylthio, mercapto, hydroxy, hydroxyalkyl, alkenyl, alkenyloxy, alkylsulfinyl, alkylsulfonyl, dialkylsulfamyl, carboxyl, carbalkoxy, carbamido, haloalkyl, cycloalkyl, cyano or alkenyloxy;
R₂ is selected from a group consisting of substituted or unsubstituted aryl, wherein said aryl group is selected from the group consisting of phenyl, pyridyl, pyrimidyl, pyrazinyl, imidazolyl, indolyl, triazinyl, tetrazolyl, thiophenyl, furanyl, thiazolyl, pyrazolyl, or pyrrolyl, and wherein said substituents are one to five independently selected from the group consisting of halogen, alkyl, alkanoyloxy, alkoxy, acylamino, amino, alkylamino, dialkylamino, dialkylaminoalkyl, sulfamyl, alkylthio, mercapto, hydroxy, hydroxyalkyl, alkenyl, alkenyloxy, alkylsulfinyl, alkylsulfonyl, dialkylsulfamyl, carboxyl, carbalkoxy, carbamido, haloalkyl, cycloalkyl, cyano or alkenyloxy;
R₃ is a substituted or unsubstituted aryl, wherein said aryl group is selected from the group consisting of phenyl, pyridyl, pyrimidyl, pyrazinyl, imidazolyl, indolyl, triazinyl, tetrazolyl, thiophenyl, furanyl, thiazolyl, pyrazolyl, or pyrrolyl, and wherein said substituents are one to five independently selected from the group consisting of halogen, alkyl, alkanoyloxy, alkoxy, acylamino, amino, alkylamino, dialkylamino, dialkylaminoalkyl, sulfamyl, alkylthio, mercapto, hydroxy, hydroxyalkyl, alkenyl, alkenyloxy, alkylsulfinyl, alkylsulfonyl, dialkylsulfamyl, carboxyl, carbalkoxy, carbamido, haloalkyl, cycloalkyl, cyano or alkenyloxy;
m is 0, 1, or 2;
n is 0, 1 or 2; and
pharmaceutically acceptable salts thereof.

22. The method of claim 21 wherein R₁ is selected from a group consisting of halogen, alkoxy, amino, dialkylamino, dialkylaminoalkyl, alkylthio, hydroxy, hydroxyalkyl, alkylsulfinyl, alkylsulfonyl, carboxyl, haloalkyl, or cyano.

23. The method of claim 22 wherein R₁ is selected from a group consisting of halogen, alkoxy, dialkylamino, dialkylaminoalkyl, alkylthio, alkylsulfinyl, alkylsulfonyl, or cyano.

24. The method of claim 21 wherein R₂ is selected from a group consisting of substituted or unsubstituted aryl, wherein said aryl group is selected from the group consisting of phenyl, pyridyl, pyrimidyl, pyrazinyl, imidazolyl, triazinyl, pyrazolyl, or pyrrolyl, and wherein said substituents are one to three independently selected from the group consisting of halogen, alkoxy, amino, alkylamino, dialkylamino, dialkylaminoalkyl, alkylthio, hydroxy, alkylsulfinyl, alkylsulfonyl, carboxyl, carbamido, haloalkyl, or cyano.

25. The method of claim 23 wherein R₂ is substituted aryl, wherein said aryl group is selected from the group consisting of phenyl, pyridyl, pyrimidyl, pyrazinyl, or triazinyl, and wherein said substituents are one or three independently selected from the group consisting of halogen, alkoxy, dialkylamino, dialkylaminoalkyl, alkylthio, alkylsulfonyl, or carboxyl.

26. The method of claim 21 where R₃ is a substituted or unsubstituted aryl, wherein said aryl group is selected from the group consisting of phenyl, pyridyl, pyrimidyl, pyrazinyl, imidazolyl, triazinyl, pyrazolyl, or pyrrolyl, and wherein said substituents are one to three independently selected from the group consisting of halogen, alkoxy, amino, dialkylamino, dialkylaminoalkyl, alkylthio, hydroxy, alkylsulfinyl, alkylsulfonyl, carboxyl, carbamido, haloalkyl, or cyano.

27. The method of claim 25 where R₃ is a substituted aryl, wherein said aryl group is selected from the group consisting of phenyl, pyridyl, pyrimidyl, pyrazinyl, triazinyl, and wherein said substituents are one or three independently selected from the group consisting of halogen, alkoxy, or, dialkylamino, dialkylaminoalkyl, alkylthio, alkylsulfonyl, or carboxyl.

28. The method of claim 21 where m is 0 or 1.

29. The method of claim 27 where m is 1.

30. The method of claim 29 where n is 1.

31. A method for inhibiting the growth of neoplastic cells comprising:
exposing the cells to a growth inhibiting effective amount of a compound of formula I 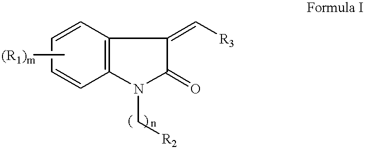
wherein
R₁ is selected from a group consisting of halogen, alkyl, alkanoyloxy, alkoxy, acylamino, amino, alkylamino, dialkylamino, dialkylaminoalkyl, sulfamyl, alkylthio, mercapto, hydroxy, hydroxyalkyl, alkenyl, alkenyloxy, alkylsulfinyl, alkylsulfonyl, dialkylsulfamyl, carboxyl, carbalkoxy, carbamido, haloalkyl, cycloalkyl, cyano or alkenyloxy;
R₂ is selected from a group consisting of substituted or unsubstituted aryl, wherein said aryl group is selected from the group consisting of phenyl, pyridyl, pyrimidyl, pyrazinyl, imidazolyl, indolyl, triazinyl, tetrazolyl, thiophenyl, furanyl, thiazolyl, pyrazolyl, or pyrrolyl, and wherein said substituents are one to five independently selected from the group consisting of halogen, alkyl, alkanoyloxy, alkoxy, acylamino, amino, alkylamino, dialkylamino, dialkylaminoalkyl, sulfamyl, alkylthio, mercapto, hydroxy, hydroxyalkyl, alkenyl, alkenyloxy, alkylsulfinyl, alkylsulfonyl, dialkylsulfamyl, carboxyl, carbalkoxy, carbamido, haloalkyl, cycloalkyl, cyano or alkenyloxy;
R₃ is a substituted or unsubstituted aryl, wherein said aryl group is selected from the group consisting of phenyl, pyridyl, pyrimidyl, pyrazinyl, imidazolyl, indolyl, triazinyl, tetrazolyl, thiophenyl, furanyl, thiazolyl, pyrazolyl, or pyrrolyl, and wherein said substituents are one to five independently selected from the group consisting of halogen, alkyl, alkanoyloxy, alkoxy, acylamino, amino, alkylamino, dialkylamino, dialkylaminoalkyl, sulfamyl, alkylthio, mercapto, hydroxy, hydroxyalkyl, alkenyl, alkenyloxy, alkylsulfinyl, alkylsulfonyl, dialkylsulfamyl, carboxyl, carbalkoxy, carbamido, haloalkyl, cycloalkyl, cyano or alkenyloxy;
m is 0, 1,or2;
n is 0, 1 or2; and
and pharmaceutically acceptable salts thereof.

32. The method of claim 31 wherein R₁ is selected from a group consisting of halogen, alkoxy, amino, dialkylamino, dialkylaminoalkyl, alkylthio, hydroxy, hydroxyalkyl, alkylsulfinyl, alkylsulfonyl, carboxyl, haloalkyl, or cyano.

33. The method of claim 32 wherein R₁ is selected from a group consisting of halogen, alkoxy, dialkylamino, dialkylaminoalkyl, alkylthio, alkylsulfinyl, alkylsulfonyl, or cyano.

34. The method of claim 31 wherein R₂ is selected from a group consisting of substituted or unsubstituted aryl, wherein said aryl group is selected from the group consisting of phenyl, pyridyl, pyrimidyl, pyrazinyl, imidazolyl, triazinyl, pyrazolyl, or pyrrolyl, and wherein said substituents are one to three independently selected from the group consisting of halogen, alkoxy, amino, alkylamino, dialkylamino, dialkylarninoalkyl, alkylthio, hydroxy, alkylsulfinyl, alkylsulfonyl, carboxyl, carbamido, haloalkyl, or cyano.

35. The method of claim 33 wherein R₂ is substituted aryl, wherein said aryl group is selected from the group consisting of phenyl, pyridyl, pyrimidyl, pyrazinyl, or triazinyl, and wherein said substituents are one or three independently selected from the group consisting of halogen, alkoxy, dialkylamino, dialkylaminoalkyl, alkylthio, alkylsulfonyl, or carboxyl.

36. The method of claim 31 where R₃ is a substituted or unsubstituted aryl, wherein said aryl group is selected from the group consisting of phenyl, pyridyl, pyrimidyl, pyrazinyl, imidazolyl, triazinyl, pyrazolyl, or pyrrolyl, and wherein said substituents are one to three independently selected from the group consisting of halogen, alkoxy, amino, dialkylamino, dialkylaminoalkyl, alkylthio, hydroxy, alkylsulfinyl, alkylsulfonyl, carboxyl, carbamido, haloalkyl, or cyano.

37. The method of claim 35 where R₃ is a substituted aryl, wherein said aryl group is selected from the group consisting of phenyl, pyridyl, pyrimidyl, pyrazinyl, triazinyl, and wherein said substituents are one or three independently selected from the group consisting of halogen, alkoxy, or, dialkylamino, dialkylaminoalkyl, alkylthio, alkylsulfonyl, or carboxyl.

38. The method of claim 31 where m is 0 or 1.

39. The method of claim 37 where m is 1.

40. The method of claim 39 where n is 1.

41. A method for regulating apoptosis in human cells comprising: exposing the cells to an effective amount of a compound of formula I 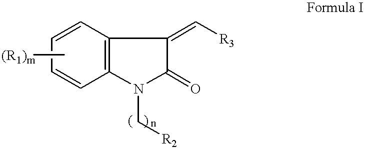
wherein
R₁ is selected from a group consisting of halogen, alkyl, alkanoyloxy, alkoxy, acylamino, amino, alkylamino, dialkylamino, dialkylaminoalkyl, sulfamyl, alkylthio, mercapto, hydroxy, hydroxyalkyl, alkenyl, alkenyloxy, alkylsulfinyl, alkylsulfonyl, dialkylsulfamyl, carboxyl, carbalkoxy, carbamido, haloalkyl, cycloalkyl, cyano or alkenyloxy;
R₂ is selected from a group consisting of substituted or unsubstituted aryl, wherein said aryl group is selected from the group consisting of phenyl, pyridyl, pyrimidyl, pyrazinyl, imidazolyl, indolyl, triazinyl, tetrazolyl, thiophenyl, furanyl, thiazolyl, pyrazolyl, or pyrrolyl, and wherein said substituents are one to five independently selected from the group consisting of halogen, alkyl, alkanoyloxy, alkoxy, acylamino, amino, alkylamino, dialkylamino, dialkylaminoalkyl, sulfamyl, alkylthio, mercapto, hydroxy, hydroxyalkyl, alkenyl, alkenyloxy, alkylsulfinyl, alkylsulfonyl, dialkylsulfamyl, carboxyl, carbalkoxy, carbamido, haloalkyl, cycloalkyl, cyano or alkenyloxy;
R₃ is a substituted or unsubstituted aryl, wherein said aryl group is selected from the group consisting of phenyl, pyridyl, pyrimidyl, pyrazinyl, imidazolyl, indolyl, triazinyl, tetrazolyl, thiophenyl, furanyl, thiazolyl, pyrazolyl, or pyrrolyl, and wherein said substituents are one to five independently selected from the group consisting of halogen, alkyl, alkanoyloxy, alkoxy, acylamino, amino, alkylamino, dialkylamino, dialkylaminoalkyl, sulfamyl, alkylthio, mercapto, hydroxy, hydroxyalkyl, alkenyl, alkenyloxy, alkylsulfinyl, alkylsulfonyl, dialkylsulfamyl, carboxyl, carbalkoxy, carbamido, haloalkyl, cycloalkyl, cyano or alkenyloxy;
m is 0, 1,or2;
n is 0, 1 or 2; and
pharmaceutically acceptable salts thereof.

42. The method of claim 41 wherein R₁ is selected from a group consisting of halogen, alkoxy, amino, dialkylamino, dialkylaminoalkyl, alkylthio, hydroxy, hydroxyalkyl, alkylsulfinyl, alkylsulfonyl, carboxyl, haloalkyl, or cyano.

43. The method of claim 42 wherein R₁ is selected from a group consisting of halogen, alkoxy, dialkylamino, dialkylaminoalkyl, alkylthio, alkylsulfinyl, alkylsulfonyl, or cyano.

44. The method of claim 41 wherein R₂ is selected from a group consisting of substituted or unsubstituted aryl, wherein said aryl group is selected from the group consisting of phenyl, pyridyl, pyrimidyl, pyrazinyl, imidazolyl, triazinyl, pyrazolyl, or pyrrolyl, and wherein said substituents are one to three independently selected from the group consisting of halogen, alkoxy, amino, alkylamino, dialkylamino, dialkylaminoalkyl, alkylthio, hydroxy, alkylsulfinyl, alkylsulfonyl, carboxyl, carbamido, haloalkyl, or cyano.

45. The method of claim 43 wherein R₂ is substituted aryl, wherein said aryl group is selected from the group consisting of phenyl, pyridyl, pyrimidyl, pyrazinyl, or triazinyl, and wherein said substituents are one or three independently selected from the group consisting of halogen, alkoxy, dialkylamino, dialkylaminoalkyl, alkylthio, alkylsulfonyl, or carboxyl.

46. The method of claim 41 where R₃ is a substituted or unsubstituted aryl, wherein said aryl group is selected from the group consisting of phenyl, pyridyl, pyrimidyl, pyrazinyl, imidazolyl, triazinyl, pyrazolyl, or pyrrolyl, and wherein said substituents are one to three independently selected from the group consisting of halogen, alkoxy, amino, dialkylamino, dialkylaminoalkyl, alkylthio, hydroxy, alkylsulfinyl, alkylsulfonyl, carboxyl, carbamido, haloalkyl, or cyano.

47. The method of claim 45 where R₃ is a substituted aryl, wherein said aryl group is selected from the group consisting of phenyl, pyridyl, pyrimidyl, pyrazinyl, triazinyl, and wherein said substituents are one or three independently selected from the group consisting of halogen, alkoxy, or, dialkylamino, dialkylaminoalkyl, alkylthio, alkylsulfonyl, or carboxyl.

48. The method of claim 41 where m is 0 or 1.

49. The method of claim 47 where m is 1.

50. The method of claim 49 where n is 1.
